(19) United States
(12) Patent Application Publication
(10) Pub. No.: US 2024/0424131 A1
(43) Pub. Date: Dec. 26, 2024

(54) TARGETING NANOPARTICLES FOR THERAPY

(71) Applicant: University of Pittsburgh - Of the Commonwealth System of Higher Education, Pittsburgh, PA (US)

(72) Inventors: Song Li, Mars, PA (US); Yixian Huang, Pittsburgh, PA (US); Yuang Chen, Pittsburgh, PA (US)

(21) Appl. No.: 18/692,919

(22) PCT Filed: Sep. 19, 2022
(86) PCT No.: PCT/US2022/043938

§ 371 Date: Mar. 18, 2024

Related U.S. Application Data
(60) Provisional application No. 63/245,810, filed on Sep. 18, 2021.
(60) Provisional application No. 63/299,431, filed on Jan. 14, 2022.

(51) Int. Cl. A61K 47/69; A61K 9/107; A61K 31/555; A61K 47/60; A61K 47/61; A61P 35/00; C12N 15/113

(52) U.S. Cl. CPC A61K 47/6907; A61K 9/1075; A61K 31/555; A61K 47/60; A61K 47/61; A61P 35/00; C12N 15/113; C12N 2310/14

(57) ABSTRACT

A formulation includes nanostructures formed from self-assembly of a plurality of amphiphilic polymers comprising cationic groups. Each of the nanostructures includes an application thereon or added thereto. The application inc hides a negatively charged targeting agent.

### CROSS-REFERENCE TO RELATED APPLICATIONS

This application claims benefit of U.S. Provisional Patent Application Ser. No. 63/245,810, filed Sep. 18, 2021, and U.S. Provisional Patent Application Ser. No. 63/299,431, filed Jan. 14, 2022, the disclosures of which are incorporated herein by reference.

### GOVERNMENTAL INTEREST

This invention was made with government support under grant numbers R01CA223788 and CA219399 awarded by the National Institutes of Health. The government has certain rights in the invention.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited herein are incorporated by reference.

Nanoparticles/nanostructures are effective in delivery or codelivery to tumors of various types of therapeutics including, for example, small molecule drugs and nucleic acids (such as siRNA). Delivery of cancer therapeutics via nanocarriers is based on the notion that tumor vasculature is leaky with fenestrae of a few to a few hundred nanometers and that long-circulating nanoparticles (NPs) selectively accumulate in the tumor tissues through a passive targeting mechanism. In addition, extravasated NPs cannot be removed from tumor tissues as a result of the compromised lymphatic system, a concept known as Enhanced Permeation and Retention (EPR) effect. While EPR is commonly seen in many syngeneic and human xenograft tumor models, it appears to be much more heterogeneous in human cancer patients. A need thus exists to develop nanocarriers capable of targeting tumors through mechanisms other than or in addition to EPR.

### SUMMARY

In one aspect, a formulation includes nanostructures formed from self-assembly of a plurality of amphiphilic polymers including cationic groups and a coating, application, or layer added to the nanostructures. The nanostructures may include an inner hydrophobic domain and an outer hydrophilic domain. The application includes a negatively charged targeting agent which targets a region of interest within a patient's body (for example, a tumor). The targeting agent may, for example, be selected from the group of a ligand for a cell receptor, a peptide, an aptamer, a polysaccharide, and an antibody. In a number of embodiments, the targeting agent is a ligand for a cell receptor (for example, a CD44 ligand).

The application may further include a hydrophilic polymeric compound. The hydrophilic polymeric compound may include a negative charge. In a number of embodiments, the hydrophilic polymeric compound includes a conjugate of a negatively charged molecule or compound and a hydrophilic polymer. The negatively charged molecule conjugated to the hydrophilic polymer may, for example, be the same compound as the targeting agent.

The formulation may further include a therapeutic compound associated with the nanostructures. The therapeutic compound may, for example, be a nucleic acid. A nucleic acid may be associated with or form a complex with the cationic groups of the nanostructures after formation thereof via charge-charge interaction.

In a number of embodiments, the therapeutic compound is a hydrophobic or lipophilic therapeutic compound. The hydrophobic or lipophilic therapeutic compound may be associated with the inner hydrophobic domain of the nanostructures. The therapeutic compound may, for example, be a small molecule therapeutic compound. The therapeutic compound may, for example, have a molecular weight below 1 kDa. In a number of embodiments, the therapeutic compound is a chemotherapeutic compound.

In a number of embodiments, the formulation further includes a second therapeutic compound, different from the therapeutic compound, wherein the second therapeutic compound includes or is a nucleic acid. As described above, the nucleic acid may be associated with the cationic groups of the amphiphilic polymers of the nanostructures.

The nucleic acid may, for example, include or be RNA or DNA. In a number of embodiments, the nucleic acid is a gene or siRNA.

As used herein, the term “cationic group” refers to an inherently cationic group or a group which forms a cation in vivo. In a number of embodiments, the group which forms a cation in vivo is an amine group, wherein the amine group is an acyclic amine group, a cyclic amine group or a heterocyclic amine group. In a number of embodiments, the amine group is selected from the group consisting of a metformin group, a morpholine group, a piperazine group, a pyridine group, a pyrrolidine group, piperidine, a thiomorpholine, a thiomorpholine oxide, a thiomorpholine dioxide, an imidazole, a guanidine. a biguanidine or a creatine.

The hydrophilic polymer may, for example, be selected from the group consisting of a polyalkylene oxide, a polyvinylalcohol, a polyacrylic acid, a polyacrylamide, a polyoxazoline, a polysaccharide and a polypeptide. In a number of embodiments, the hydrophilic polymer is polyethylene glycol.

A ratio of the negatively charged targeting agent to the hydrophilic polymeric compound added to the nanostructures may be determined such that uptake of the nanostructures at one or more regions other than the region of interest is maintained at a sufficiently low level to allow interaction of the negatively charged targeting agent at the region of interest (for example, a tumor).

In a number of embodiments, each of the plurality of amphiphilic polymers includes a hydrophobic polymer backbone, a first plurality of pendant groups attached to the hydrophobic polymer backbone and including at least one of the cationic groups, and a second plurality of pendant groups attached to the hydrophobic polymer backbone and including at least one hydrophilic polymer (that is, a pendant hydrophilic polymer). The hydrophobic polymer backbone may, for example, further include a pendant lipidic group.

In a number of embodiments, the hydrophobic polymer backbone is formed via a free radical polymerization. The hydrophobic polymer backbone may, for example, be formed via a controlled/living radical polymerization or a reversible-deactivation radical polymerization.

In another aspect, a method of formulating a composition includes forming nanostructures via self-assembly of a plurality of amphiphilic polymers including cationic groups in an aqueous medium and adding or applying a coating, application, or layer to the nanostructures by adding to the nanostructures a negatively charged targeting agent.

In another aspect, a method of delivering a therapeutic compound to a patient includes administering a formulation including nanostructures formed from self-assembly of a plurality of amphiphilic polymers including cationic groups. Each of the nanostructures includes a coating, application, or layer on the nanostructures. The application includes a negatively charged targeting agent.

In a number of embodiments, the targeting agent is a CD44 ligand. In that regard, in one aspect, a formulation includes nanostructures formed from self-assembly of a plurality of amphiphilic polymers comprising cationic groups. The nanostructures include an application added thereto. The application includes a negatively charged CD44 ligand and a hydrophilic polymeric compound. The hydrophilic polymeric compound may include a negative charge. In a number of embodiments, the hydrophilic polymeric compound is a conjugate of a negatively charged molecule with a hydrophilic polymer. The negatively charged molecule, which is conjugated to the hydrophilic polymer. may, for example, be a CD44 ligand. In a number of embodiments, the hydrophilic polymeric compound is a conjugate of a hydrophilic polymer and the CD44 ligand that is present in the coating.

The nanostructures may further include an inner hydrophobic domain and an outer hydrophilic domain. The nanostructures may, for example, be micelles. The CD44 ligand(s) hereof may, for example, include osteopontin, a collagen, a matrix metalloproteinase. chondroitin sulfate, hyaluronic acid, or a derivative of such ligands (which remains active as a CD44 ligand). In a number of embodiments, the CD44 ligand is chondroitin sulfate or hyaluronic acid. In a number of embodiments, the CD44 ligand is chondroitin sulfate.

The formulation may further include a therapeutic compound associated with the nanostructures. The therapeutic compound may, for example, include a nucleic acid which is added to the nanostructures before application of the negatively charged CD44 ligand and the hydrophilic polymeric compound, which may, for example, be applied as a mixture. The therapeutic compound may, for example, include a hydrophobic or lipophilic therapeutic compound. In a number of embodiments, the hydrophobic or lipophilic therapeutic compound is a small molecule therapeutic compound. The small-molecule therapeutic compound may, for example, have a molecular weight below 1 kDa.

In a number of embodiments, the therapeutic compound (or first therapeutic compound) is a hydrophobic or lipophilic therapeutic compound and the formulation further includes a second therapeutic compound, different from the therapeutic compound, wherein the second therapeutic compound comprises a nucleic acid. As described above, the therapeutic compound (or first therapeutic compound) may be a small molecule therapeutic compound.

The nucleic acid of formulations hereof may, for example, include RNA or DNA. In a number of embodiments, the nucleic acid is a gene or siRNA. In a number of embodiments, the nucleic acid is siRNA.

The cationic groups hereof may include an inherently cationic group or a group which forms a cation in vivo. In a number of embodiments wherein the group forms a cation in vivo, the cationic group is an amine group, wherein the amine group is an acyclic amine group, a cyclic amine group or a heterocyclic amine group. The amine group may, for example, be selected from the group consisting of a metformin group, a morpholine group, a piperazine group, a pyridine group, a pyrrolidine group, piperidine, a thiomorpholine, a thiomorpholine oxide, a thiomorpholine dioxide, an imidazole, a guanidine, a biguanidine or a creatine. In a number of embodiments, the amine group is a biguanidine.

The hydrophilic polymer may, for example, be selected from the group consisting of a polyalkylene oxide, a polyvinylalcohol, a polyacrylic acid, a polyacrylamide, a polyoxazoline, a polysaccharide and a polypeptide. In a number of embodiments, the hydrophilic polymer is polyethylene glycol.

In a number of embodiments, a ratio of the negatively charged CD44 ligand to the hydrophilic polymeric compound is such or determined such that uptake of the nanostructures in the liver of a patient is maintained at a sufficiently low level to allow interaction of the negatively charged CD44 ligand with CD44 on a tumor remote from the liver.

In a number of embodiments, each of the plurality of amphiphilic polymers includes a hydrophobic polymer backbone, a first plurality of pendant groups attached to the hydrophobic polymer backbone and comprising at least one of the cationic groups, and a second plurality of pendant groups attached to the hydrophobic polymer backbone and comprising at least one hydrophilic polymer. The pendant hydrophilic polymer may, for example, be selected from the group consisting of a polyalkylene oxide, a polyvinylalcohol, a polyacrylic acid, a polyacrylamide, a polyoxazoline, a polysaccharide and a polypeptide. In a number of embodiments, the pendant hydrophilic polymer is polyethylene glycol. The hydrophobic polymer backbone may further include a pendant lipidic group.

In a number of embodiments, the hydrophobic polymer backbone is formed via a free radical polymerization. The hydrophobic polymer backbone may, for example, be formed via a reversible-deactivation radical polymerization.

In another aspect, a method of formulating a composition includes forming nanostructures via self-assembly of a plurality of amphiphilic polymers including cationic groups in an aqueous medium and adding or creating a coating on an exterior of each of the nanostructures by adding to the nanostructures a negatively charged CD44 ligand and a hydrophilic polymeric compound. The hydrophilic polymeric compound may include a negative charge. In a number of embodiments, the hydrophilic polymeric compound includes or is a conjugate of a negatively charged molecule and a hydrophilic polymer. The negatively charged molecule which is conjugated to the hydrophilic polymer may, for example, be a CD44 ligand. In a number of embodiments, the hydrophilic polymeric compound is a conjugate of a hydrophilic polymer and the CD44 ligand that is present in the coating.

As described above, each of the nanostructures may, for example, include an inner hydrophobic domain and an outer hydrophilic domain. The nanostructures may, for example, be micelles. The CD44 ligand(s) hereof may, for example, include osteopontin, a collagen, a matrix metalloproteinase, chondroitin sulfate, hyaluronic acid, or a derivative of such ligands (wherein the derivative retains activity as a CD44 ligand). In a number of embodiments, the CD44 ligand is chondroitin sulfate or hyaluronic acid. In a number of embodiments, the CD44 ligand is chondroitin sulfate.

The method may further include associating a therapeutic compound with the nanostructures. The therapeutic compound may, for example, include a nucleic acid which is added to the nanostructures before application of the negatively charged CD44 ligand and the hydrophilic polymeric compound. The negatively charged CD44 ligand and the hydrophilic polymeric compound may, for example, be applied as a mixture. The therapeutic compound may, for example, include or be a hydrophobic or lipophilic therapeutic compound. In a number of embodiments, the hydrophobic or lipophilic therapeutic compound is a small molecule therapeutic compound. The small-molecule therapeutic compound may, for example, have a molecular weight below 1 kDa.

In a number of embodiments, the therapeutic compound is a hydrophobic or lipophilic therapeutic compound and the formulation further includes a second therapeutic compound, different from the therapeutic compound, wherein the second therapeutic compound includes a nucleic acid. As described above, the therapeutic compound may be a small molecule therapeutic compound.

The nucleic acid of formulations hereof may, for example, include RNA or DNA. In a number of embodiments, the nucleic acid is a gene or siRNA. In a number of embodiments, the nucleic acid is siRNA.

The cationic groups hereof may include an inherently cationic group or a group which forms a cation in vivo. In a number of embodiments wherein the group forms a cation in vivo, the cationic group is an amine group, wherein the amine group is an acyclic amine group, a cyclic amine group or a heterocyclic amine group. The amine group may, for example, be selected from the group consisting of a metformin group, a morpholine group, a piperazine group, a pyridine group, a pyrrolidine group, piperidine, a thiomorpholine, a thiomorpholine oxide, a thiomorpholine dioxide, an imidazole, a guanidine, a biguanidine or a creatine. In a number of embodiments, the amine group is a biguanidine.

The hydrophilic polymer may, for example, be selected from the group consisting of a polyalkylene oxide, a polyvinylalcohol, a polyacrylic acid, a polyacrylamide, a polyoxazoline, a polysaccharide and a polypeptide. In a number of embodiments, the hydrophilic polymer is polyethylene glycol.

In a number of embodiments, a ratio of the negatively charged CD44 ligand to the hydrophilic polymeric compound is determined such that uptake of the nanostructures in the liver of a patient is maintained at a sufficiently low level to allow interaction of the negatively charged CD44 ligand with CD44 on a tumor remote from the liver.

In a number of embodiments, each of the plurality of amphiphilic polymers includes a hydrophobic polymer backbone, a first plurality of pendant groups attached to the hydrophobic polymer backbone and including at least one of the cationic groups, and a second plurality of pendant groups attached to the hydrophobic polymer backbone and including at least one hydrophilic polymer. The pendant hydrophilic polymer may, for example, be selected from the group consisting of a polyalkylene oxide, a polyvinylalcohol, a polyacrylic acid, a polyacrylamide, a polyoxazoline, a polysaccharide and a polypeptide. In a number of embodiments, the hydrophilic polymer is polyethylene glycol. The hydrophobic polymer backbone may further include a pendant lipidic group.

In a number of embodiments, the hydrophobic polymer backbone is formed via a free radical polymerization. The hydrophobic polymer backbone may, for example, be formed via a reversible-deactivation radical polymerization.

In a further aspect, a method of delivering a therapeutic compound to a patient includes administering a formulation including nanostructures formed from self-assembly of a plurality of amphiphilic polymers including cationic groups and a coating on an exterior of each of the nanostructures. The coating includes a CD44 ligand and a hydrophilic polymeric compound, the therapeutic compound being associated with the formulation. As described above, the nanostructures may further include an inner hydrophobic domain and an outer hydrophilic domain. The nanostructures may, for example, be micelles. The nanostructures may be further characterized as described above and elsewhere herein.

The present devices, systems, methods, and compositions, along with the attributes and attendant advantages thereof, will best be appreciated and understood in view of the following detailed description taken in conjunction with the accompanying drawings.

### BRIEF DESCRIPTION OF THE DRAWINGS

FIG. 1 illustrates the representative example of codelivery of TCF4 siRNA and a prodrug conjugate of 5-FU with OXP (FuOXP) in a murine syngeneic CRC (CT26) model, wherein N=5, **P<0.01, and ***P<0.001.

FIG. 2A illustrates schematically an embodiment of a methodology for formation of representative nanostructures or nanoparticles of a nanocarrier hereof for delivery of, for example, a nucleic acid (siRNA) and/or a small molecule therapeutic drug (FuOXP) including a targeting agent such as the CD44 ligand chondroitin sulfate (CS).

FIG. 2B illustrates a table setting forth sizes, zeta potential, drug loading content (DLC), and drug loading efficiency (DLE) of PMAOB/FuOXP mixed micelles at various carrier/drug ratios (w/w).

FIG. 2C illustrates gel retardation assays showing that stable complexes were formed at nitrogen (N)/phosphate (P) ratios (ratio of positively-chargeable polymer amine (N=nitrogen) groups to negatively-charged nucleic acid phosphate (P) groups) of 1 and above.

FIG. 2D illustrates sizes and zeta potentials of FuOXP/siRNA-coloaded micelles at different N/P ratios.

FIG. 2E illustrates sizes and zeta potentials of FuOXP/siRNA-coloaded micelles at different N/P/sulphate(S) ratios.

FIG. 2F illustrates sizes and zeta potentials of FuOXP/siRNA-coloaded micelles at different N/P/S (CS)/S (CS-PEG) ratios.

FIG. 2G illustrates a study of change in NP size after a period of two weeks in PBS and room temperature and in mouse serum (MS) after 24 hours.

FIG. 2H illustrates gel retardation studies demonstrating that free siRNA was completely degraded following treatment with RNAse III at 37° C. for 1 h while siRNA loaded into PMAOB-CP NPs was well protected from the degradation by RNAse III.

FIG. 3A illustrates an embodiment of a synthesis route for the polymer PMAOB.

FIG. 3B illustrates a study of size and zeta potential of micelle/siRNA complexes (w/o FuOXP) at various N/P ratios.

FIG. 3C illustrates studies of the tissue distribution of Cy5-labeled siRNA in the liver and a tumor for various CS/CS-PEG rations.

FIG. 4A illustrates whole-body NIR imaging of a tumor-bearing mouse demonstrating that Cy5 signals were concentrated in the tumor areas (subcutaneous (s.c.) CT26 model).

FIG. 4B illustrates er vivo images of the heart, kidney, spleen, lung, liver, and a tumor.

FIG. 4C illustrates quantitative fluorescence intensity over 48 hours in the liver and a tumor.

FIG. 4D illustrates images of Cy5-labeled siRNA in NPs and Cy5-labeled free siRNA in blood over time.

FIG. 4E illustrates NIR images of the heart, kidney, spleen, lung, liver and a tumor in human colon cancer (WiDr), human breast cancer (BT-474), murine pancreatic cancer (Panc02), and murine breast cancer (4T1.2) models.

FIG. 4F illustrates tissue distribution studies in an orthotopic murine colon cancer model.

FIG. 4G illustrates the distribution of Cy5 siRNA in tumor sections at 24 h following i.v. administration of siRNA NPs.

FIG. 5A illustrates whole-body NIR imaging showing the distribution of Cy5-labeled siRNA in tumors in wild-type (WT) and CD44⁻⁻ mice.

FIG. 5B illustrates ex vivo imaging of the liver and a tumor from WT and CD44^{-/31} mice.

FIG. 5C illustrates images of Cy5-labeled siRNA in serum from WT and CD44^{-/31} mice.

FIG. 5D illustrates quantitative intensity of Cy5 signals in tumor and liver tissues in wild type (WT) CD44 mice and CD44^{-/31} mice.

FIG. 5E illustrates quantitative intensity of Cy5 signals in blood in wild type (WT) CD44 mice and CD44^{-/31} mice.

FIG. 5F illustrates whole body NIR imaging of WT and Zombie mice in which the passive targeting mechanism such as EPR remains effective while the active transendothelial transport mechanism is inhibited.

FIG. 5G illustrates quantitative intensity of Cy5 signals in tumor and liver tissues in wild type (WT) mice and Zombie mice.

FIG. 5H illustrates a study of NP uptake as a function of CS/PEG-CS ratio in mouse liver sinusoidal endothelial cells (LSECs) and human umbilical vein endothelial cells (HUVECs).

FIG. 5I illustrates a study of the role of transcytosis in tumor targeting by NPs hereof wherein significant transfection was found in CT26 plated in a lower chamber of a Transwell when Cy5 siRNA NPs were applied to HUVECs in the upper chamber and that transfection of Ct26 cells was significantly inhibited by dynasore (NP+1), an endocytosis inhibitor, indicating the effectiveness of NPs hereof in mediating transcytosis through vascular ECs.

### DETAILED DESCRIPTION

It will be readily understood that the components of the embodiments, as generally described and illustrated in the figures herein, may be arranged and designed in a wide variety of different configurations in addition to the described representative embodiments. Thus, the following more detailed description of the representative embodiments, as illustrated in the figures, is not intended to limit the scope of the embodiments, as claimed, but is merely illustrative of representative embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “a,” “an”, and “the” include plural references unless the context clearly dictates otherwise. Thus, for example, reference to “a therapeutic compound” includes a plurality of such therapeutic compounds and equivalents thereof known to those skilled in the art, and so forth, and reference to “the therapeutic compound” is a reference to one or more such therapeutic compounds and equivalents thereof known to those skilled in the art, and so forth. Recitation of ranges of values herein are merely intended to serve as a shorthand method of referring individually to each separate value falling within the range. Unless otherwise indicated herein, and each separate value, as well as intermediate ranges, are incorporated into the specification as if individually recited herein. All methods described herein can be performed in any suitable order unless otherwise indicated herein or otherwise clearly contraindicated by the text.

As used herein, the term “polymer” refers to a chemical compound that is made of a plurality of small molecules or monomers that are arranged in a repeating structure to form a larger molecule. Polymers may occur naturally or be formed synthetically. The use of the term “polymer” encompasses homopolymers as well as copolymers. The term “copolymer” is used herein to include any polymer having two or more different monomers. Copolymers may, for example, include alternating copolymers, periodic copolymers, statistical copolymers, random copolymers, block copolymers, graft copolymers etc. Examples of polymers include, for example, polyalkylene oxides.

As used herein, the term “pendant” refers to a group or moiety attached to a backbone chain of a long molecule such as a polymer as described above. Pendant group may be either (1) short chain or low molecular weight groups or (2) long chain or high molecular groups such as polymers. Pendant groups are sometime referred to as side groups. Long chain pendant groups or high molecular weight pendant groups are sometimes referred to as “pendant chains” or “side chains”.

In a number of embodiments, the systems, formulations, methods, and compositions hereof are used in delivery and/or co-delivery of small molecule therapeutic agents or drugs (for example, chemotherapeutic therapeutic agents or drugs) and/or nucleic acid-based therapeutic agents or drugs. The amphiphilic polymer may, for example, be formed via radical polymerization to have a hydrophobic polymer backbone. The hydrophobic polymer backbone may, for example, be formed via a free radical polymerization or via a reversible-deactivation radical polymerization or RDRP (formerly referred to a controlled radical polymerization or CRP).

Reversible-Deactivation Radical Polymerization (RDRP) procedures include, for example, Nitroxide Mediated Polymerization (NMP), Atom Transfer Radical Polymerization (ATRP), and Reversible Addition Fragmentation Transfer (RAFT) and others (including cobalt mediated transfer) that have evolved over the last two decades. RDRP provide access to polymers and copolymers including radically polymerizable/copolymerizable monomers with predefined molecular weights, compositions, architectures and narrow/controlled molecular weight distributions. Because RDRP processes can provide compositionally homogeneous well-defined polymers, with predicted molecular weight, narrow/designed molecular weight distribution, and high degrees of α-and ω-chain end-functionalization, they have been the subject of much study, as reported in several review articles and ACS symposia. See, for example, Qiu, J.; Charleux, B.; Matyjaszewski, K., Prog. Polym. Sci. 2001, 26, 2083; Davis, K. A.; Matyjaszewski, K. Adv. Polym. Sci. 2002, 159, 1; Matyjaszewski, K., Ed. Controlled Radical Polymerization; ACS: Washington, D. C., 1998: ACS Symposium Series 685. Matyjaszewski, K., Ed.; Controlled/Living Radical Polymerization. Progress in ATRP, NMP, and RAFT; ACS: Washington, D. C., 2000; ACS Symposium Series 768; and Matyjaszewski, K., Davis, T. P., Eds. Handbook of Radical Polymerization; Wiley: Hoboken, 2002, the disclosures of which are incorporated herein by reference.

The hydrophobic polymer backbone may be formed via radical polymerization of radically polymerizable monomers (including conventional or free radical polymerization as well as RDRP). Such monomers may include pendant groups prior to polymerization. Alternatively, such pendant groups may be attached after polymerization. Representative monomers for use herein include styrene, acrylic acid, methacrylic acid, acrylonitrile, vinyl monomers and their derivatives. In a number of embodiments, the degree of polymerization for hydrophobic polymers hereof is, for example, less than 500.

In a number of embodiments, the polymers further include a first plurality of pendant groups attached to the hydrophobic polymer backbone and including at least one cationic group and a second plurality of pendant groups attached to the hydrophobic polymer backbone and including at least one hydrophilic polymer (as described above). Pendant group hereof may also include both at least one cationic group and at least one hydrophilic polymer. In a number of embodiments, at least one of the first plurality of pendant groups and the second plurality of pendant groups is attached to the hydrophobic polymer backbone via a linking moiety.

As set forth above, the at least one cationic group may, for example, include an inherently cationic group or a group which forms a cation in the formulations hereof and/or in vivo (for example, an amine group which forms a cation in vivo). The amine group may be an acyclic amine group, a cyclic amine group or a heterocyclic amine group. The at least one cationic group may, for example, be selected from the group consisting of a biguanidine group. a metformin group, a morpholine group, a piperazine group, a pyrrolidine group, a piperidine group, a thiomorpholine, a thiomorpholine oxide, a thiomorpholine dioxide, imidazole, guanidine, or creatine. In a number of embodiments, the at least one cationic group is selected from the group consisting of a metformin group, a morpholine group, a piperazine group or creatine. The cationic amine groups described herein may be substituted or unsubstituted.

Pendant groups hereof may, for example, be attached to the hydrophobic polymer backbone via a linking moiety that is labile under in vivo conditions (for example, under acidic pH conditions). The labile bond may, for example, be sensitive to conditions in a target region (for example, sensitive to or labile under acidic conditions in the region of a tumor). An acid-labile bond may, for example, include a carboxydimethyl maleate, a hydrazine, an imine, an acetal, an oxime, a silyl ether, a cis-asonityl or another acid-labile bond or linkage. Use of a labile bond that is sensitive to acidic conditions may, for example, be used to cleave the hydrophilic polymer/oligomer in, for example, an acidic tumor environment. Examples of other suitable labile bonds include disulfide bonds, hypoxia sensitive bonds and glucose-sensitive bonds.

Hydrophilic oligomers or hydrophilic polymers hereof may, for example, be selected from the group consisting of a polyalkylene oxide, a polyvinylalcohol, a polyacrylic acid, a polyacrylamide, a polyoxazoline, a polysaccharide and a polypeptide. In a number of embodiments, the at least one hydrophilic polymer is a polyalkylene oxide. The polyalkylene oxide may, for example, be a polyethylene glycol. A polyethylene glycol or other hydrophilic polymer hereof may, for example, have a molecular weight of at least 500 Da. In a number of embodiments, the polyethylene glycol of other hydrophilic polymer hereof has a molecular weight in the range of 200 Da to 10 Kda or a range of 500 to 5 Kda.

In a number of embodiments, a formulation or nanocarrier formulation includes nanostructures or nanoparticles formed from self-assembly (in an aqueous medium) of a plurality of amphiphilic polymers including cationic groups. The nanostructures may, for example, include an inner hydrophobic domain and an outer hydrophilic domain. The nanostructure may likewise include a coating, application, or layer on an outer region or exterior region of the nanostructures. The coating, application, or layer need not be continuous. In a number of embodiments, the coating, application, or layer includes a negatively charged targeting agent. As used herein, the term “targeting agent” refers generally to an agent which actively targets a region of interest such as a tumor. The negative charge of the targeting agent provides anchoring via charge-charge interactions with cationic groups of the amphiphilic polymers forming the nanostructures. In addition to providing targeting, such negatively charged agents assist in charge neutralization/shielding of positive charge to achieve a nanostructure/nanocarrier exhibiting approximate charge neutrality. The coating, application, or layer may further include a hydrophilic polymeric compound which may, for example. inchide a negative charge to anchor the hydrophilic polymeric compound to the cationic groups via charge-charge interaction. The hydrophilic polymeric compound may provide further charge neutralization and may, in some, embodiments, provide a degree of shielding for the targeting agent as further discussed below.

In a number of embodiments, the negatively charged targeting agent includes or is a ligand for a cell receptor, a peptide, an aptamer, a polysaccharide, and an antibody. The negative charge may be inherent in the targeting agent or be added thereto (for example, via conjugation with a negatively charged molecule). In a number of representative studies hereof the targeting agent is a negatively charged CD44 ligand. The application may, for example, include a negatively charged CD44 ligand and a hydrophilic polymeric compound as described above. Examples of suitable CD44 ligands include osteopontin, a collagen, a matrix metalloproteinase, chondroitin sulfate (CS), hyaluronic acid (HA), or derivatives of such ligands (which retain targeting activity). As also described above, the hydrophilic polymeric compound may include a negative charge. The hydrophilic polymeric compound may be formed by conjugating a negatively charged molecule such as chondroitin sulfate or CS with a hydrophilic polymer as described above. In, for example, a CS-PEG conjugate, CS provides negative charge to interact via charge-charge interaction with positive charges associated with the cations of the amphiphilic polymer of the nanostructures. In general, any compound with suitable negative charge can be conjugated with a hydrophilic polymer such as PEG to anchor the hydrophilic polymer conjugate to the nanostructure. Such a compound can, for example, be another negatively charged CD44 ligand, a bio-compound, a synthetic compound, etc. Alternatively, a portion of the hydrophilic polymer may be modified to include a negative charge. In a number of embodiments, the hydrophilic polymeric compound is a conjugate of a hydrophilic polymer and the CD44 ligand that is present in the application.

The nanocarrier formulations hereof may, for example, be used to deliver therapeutic compounds that associate with or interact with, for example, the hydrophobic domain and/or with the cationic groups of the nanocarrier. The nanocarrier formulations hereof are, for example, capable of delivery or codelivery of small molecule, hydrophobic or lipophilic therapeutic compounds or drugs and/or nucleic acids (for example, siRNA, genes, plasmids, etc.). The incorporation of nucleic acids in nanostructures or nanoparticles formed from polymers including cationic groups is, for example, described in Published U.S. Patent Application No. 2021/0236645, the disclosure of which is incorporated herein by reference. Multivalent charge-charge interactions between the cationic groups of the amphiphilic polymer molecules and nucleic acids may serve as a simple approach to create interactive, non-covalent crosslinks between amphiphilic polymeric molecules of the micelles hereof.

The nanocarrier formulations hereof, which are coated with CD44 ligands, are highly effective in tumor targeting through both EPR and transcytosis through tumor endothelial cells. In a number of embodiments, such negatively charged ligands may assist in stabilizing micelles. Such nanocarriers were characterized with respect to both biophysical properties and the efficiency of tumor targeting.

Moreover, non-covalent interactions such as π-π stacking (for example, via inclusion of aromatic group), hydrogen bonding, etc. between the amphiphilic polymer molecules and nucleic acids may additionally be used to create interactive, non-covalent interactions between amphiphilic polymeric molecules of the micelles hereof and therapeutic compound(s), π-π and hydrophobic interactions or stacking and other interactions between the groups of amphiphilic polymers forming nanostructures/micelles and numerous compounds such as drugs are, for example, discussed in U.S. Pat. Nos. 10,172,795 and 9,855,341 and U.S. Patent Publication Nos. 2018/0214563 and 2021/0236645, the disclosures of which are incorporated herein by reference.

In a number of studies, the nanocarriers hereof were, for example, demonstrated to be highly effective in codelivery of a nucleic acid such as siRNA and a drug such as a chemotherapeutic drugs. Immunotherapy is among the most rapidly evolving strategies in cancer treatment. In particular, immune checkpoint blockade (ICB) using inhibitors of PD-1and/or CTLA-4 has clearly shown its therapeutic potential in clinic. However, only a small population of patients benefit from this treatment. There is an urgent need to develop novel therapies targeting other immune checkpoints to benefit more cancer patients.

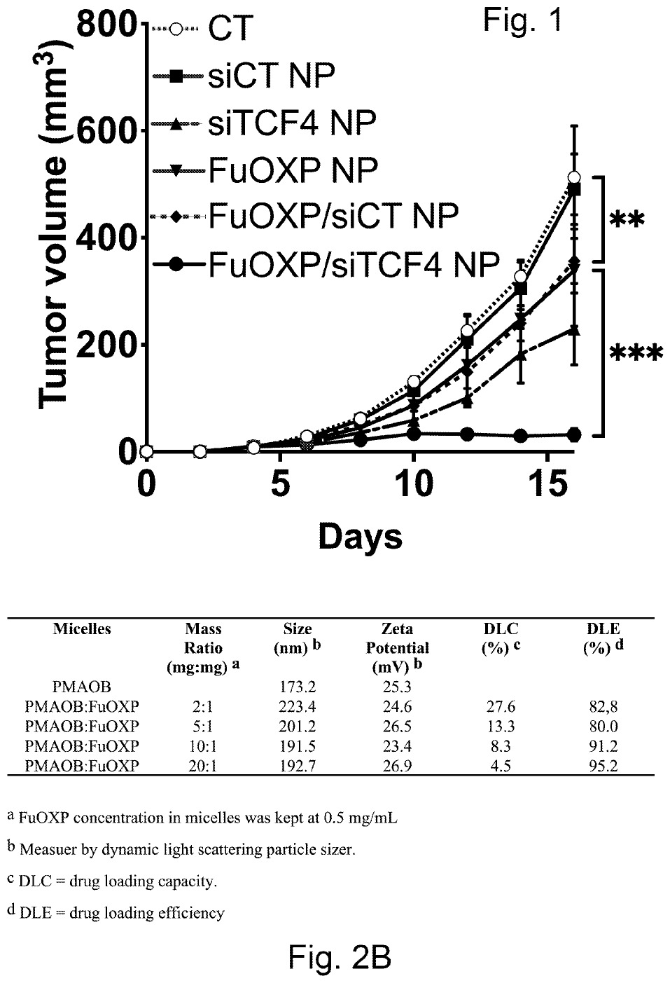

The therapeutic efficacy as well as the underlying mechanism of codelivery of representative siRNA and the representative chemotherapeutic drug 5-Fu/cisplatin were studied using nanocarriers hereof in various cancer models. 5-FU and OXP are the front-line therapeutic agents for colorectal cancer (CRC), and the major treatment for patients with various stages of pancreatic cancer (PCa) including advanced or metastatic PCa but are associated with issues of limited efficacy and systemic toxicity. Previously, a lipid-derivatized prodrug conjugate of 5-FU and cisplatin (fuplatin) was reported to have improved antitumor activity and decreased cytotoxicity towards normal cells. In a number of studies hereof, a prodrug conjugate of 5-FU with OXP (FuOXP) was similarly synthesized as OXP shows superiority over cisplatin in clinic. CT26 is a syngeneic CRC model that responded poorly to moderately to 5-FU/OXP as well as FuOXP (data not shown). In a number of studies, RNAseq of CT26 tumors was conducted following treatment with FuOXP every 5 days for 3 times and TCF4 was one of the genes that were significantly induced (data not shown). FIG. 1, for example, illustrates the representative example of codelivery of TCF4 siRNA and FuOXP, which led to significant inhibition of tumor growth in a murine syngeneic CRC (CT26) model. Transcription Factor 4 (TCF4) is an oncoprotein and is involved in the oncogenesis and drug resistance in CRC. As described above, mice bearing CT26 tumors received various treatments once every 5 days for 3 times at a siRNA dose of 1 mg/kg and FuOXP dose of 5 mg/kg. FuOXP nanoparticles (NPs) alone slightly inhibited the growth of CT26 tumor. TCF4 siRNA NPs alone showed modest effect in controlling the growth of the tumor. However, combination of both led to significant improvement in the antitumor activity. Tumor volumes were followed once every 2 days.

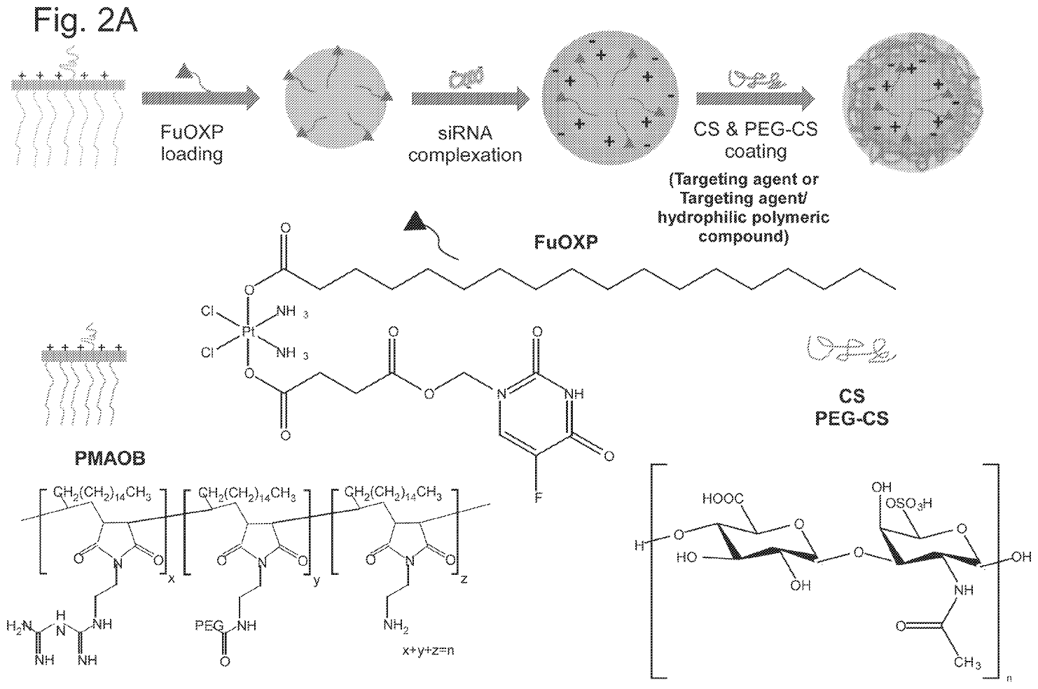

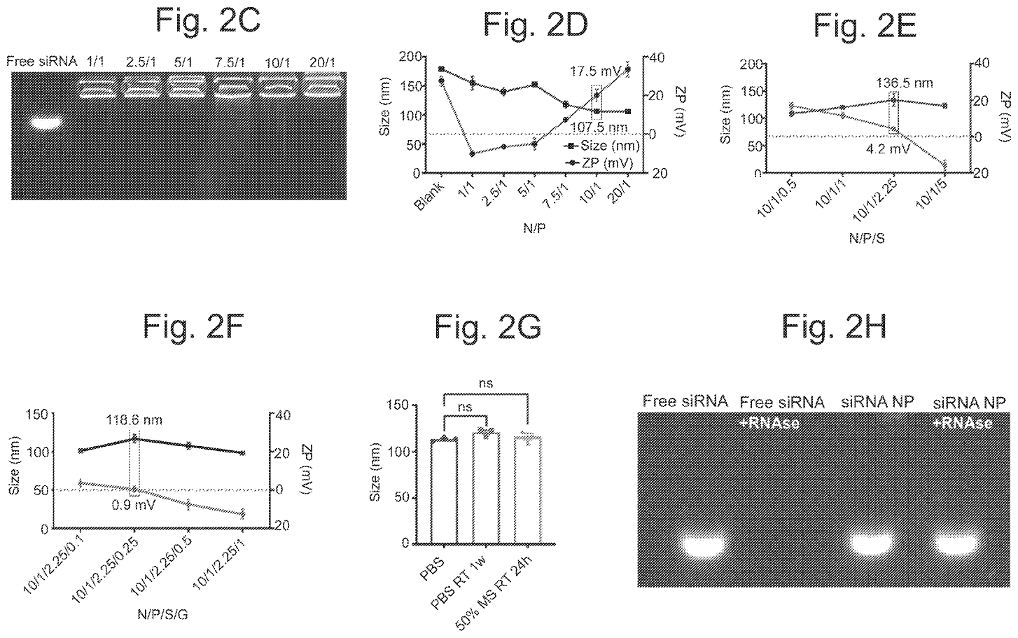

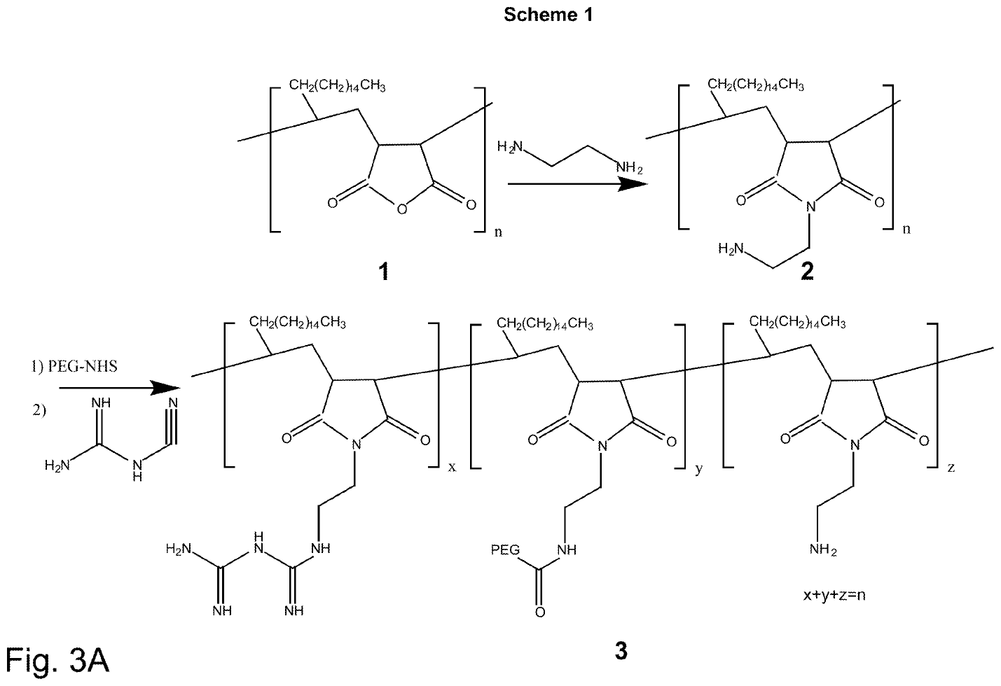

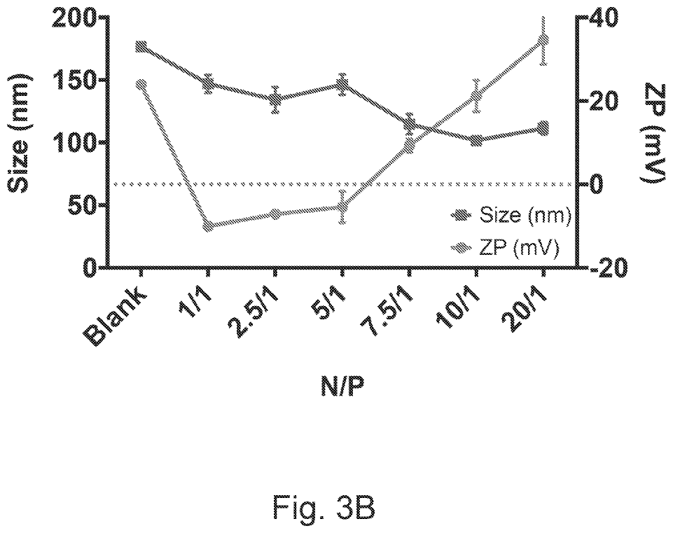

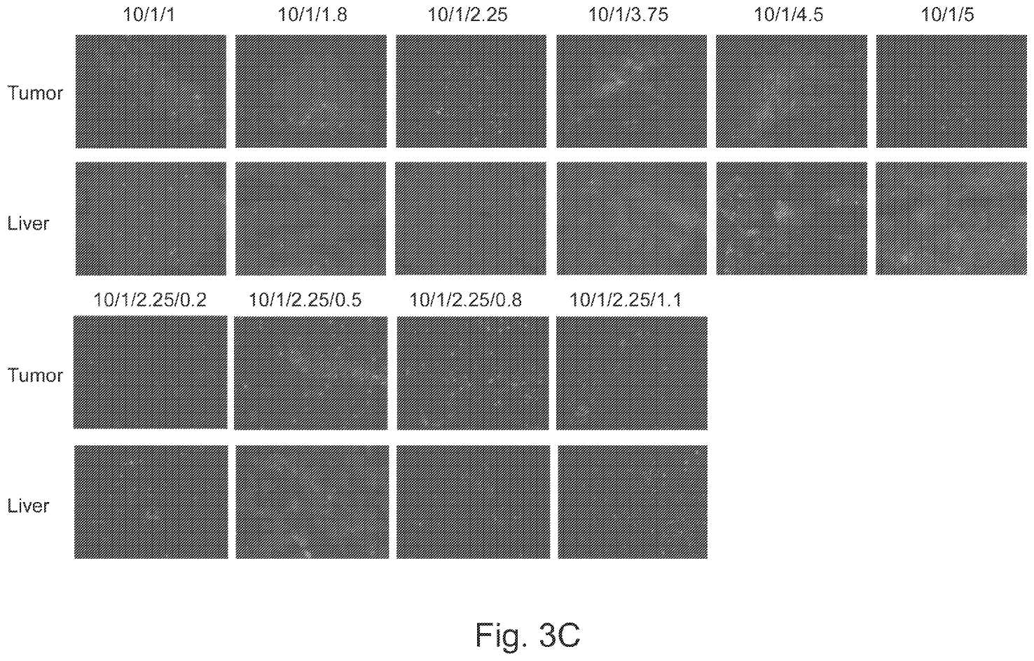

In a number of embodiments hereof, a nanocarrier formed from a representative poly (maleic anhydride-alt-1-octadecene or PMAO polymer (PMAOB-CP) was developed to achieve codelivery of siRNA and FuOXP (see, for example, FIGS. 2A through 2H), FIG. 2A shows the major components and steps in the development of the PMAOB-CP nanocarrier. PMAOB is an amphiphilic polymer that self-assembles to form micelles in aqueous solutions. The lipid motif could facilitate the interaction with cell membrane and improve transfection. It also helps to improve the loading of FuOXP into the hydrophobic/lipophilic core. The biguanidine motif was designed to enhance the interaction with siRNA as a result of its highly positive nature. The synthesis route of PMAOB is shown in Scheme 1 of FIG. 3A. Poly (maleic anhydride-alt-1-octadecene or PMAO (compound 1; a commercially available polymer) was first reacted with ethylenediamine to introduce amine groups. The amine-containing PMAO polymer (compound 2) was then sequentially reacted with PEG_{2K}-NHS and dicyandiamide to introduce PEG and biguanide pendant groups, respectively. The nuclear magnetic resonance (¹H NMR) spectra of PMAOB (compound 3) in DMSO showed the respective PMAO methyl peaks (1.0-1.2 ppm), and PEG methyl peaks (3.28 ppm) and methylene peaks (3.3-3.6 ppm). The PEG substitution was ˜10% and all other amine groups of compound 2 were derivatized with biguanide groups based on the ninhydrin assay.

PMAOB polymer readily formed micelles in PBS with a size of 173.2 nm. FuOXP could be loaded into PMAOB micelles at a carrier/drug ratio as low as 2/1. FIG. 2B shows the sizes of PMAOB/FuOXP mixed micelles at various carrier/drug ratios (w/w). PMAOB/FuOXP mixed micelles at a carrier/drug ratio of 10/1 were used for further complexation with siRNA.

Both drug-free and FuOXP-loaded PMAOB micelles readily formed complexes with siRNA in aqueous solutions. Gel retardation assay showed that stable complexes were formed at nitrogen (N)/phosphate (P) ratios of 1 and above (FIG. 2C). FIG. 2D shows the sizes and zeta potentials of FuOXP/siRNA-coloaded micelles at different N/P ratios. Increasing the N/P ratio from 1/1 to 2.5/1 was associated with a decrease in the size of the complexes, and the complexes were negative in Zeta potentials. There was an increase in the size of the complexes upon increasing the N/P ratio to 5/1. This increase is likely due to charge neutrality at this ratio, which tends to cause aggregation of the complexes. Further increases in N/P ratio led to formation of complexes with gradual increases in Zeta potentials and decreases in sizes. At a N/P ratio of 10/1, the resulting FuOXP/siRNA/PMAOB complexes were positively charged (17.5 mV) and 107.5 nm in size, smaller than that of PMAOB micelles loaded with FuOXP alone (179.1 nm) (FIG. 2D), indicating that siRNA was able to cross-link and stabilize the micelles. Similar results were shown when siRNA formed complexes with drug-free PMAOB micelles (see FIG. 3B), indicating a PMAOB-based carrier hereof can be used for delivery of siRNA alone or codelivery of siRNA and FuOXP.

It is well known that carriers with cationic surface are not suitable for systemic delivery to distant solid tumors despite their potential in delivery to pulmonary vasculature including lung metastasis. Therefore, in representative studies, the stable FuOXP/siRNA-coloaded micelles formed at a N/P ratio of 10/1 were subjected to surface coating/application with a mixture of chondroitin sulfate (CS) and a representative hydrophilic compound in the form of a CS-PEG conjugate. CS is a highly charged molecule and CS/CS-PEG were used to decrease the surface positive charge of the resulting NPs (PMAOB-CP NPs). In addition, small amounts of PEG were included to minimize the nonspecific interaction with serum proteins. As shown in FIG. 2E, increasing the amounts of CS led to gradual neutralization of positive charges. At a N/P/sulphate(S) ratio of 10/1/2.25, the resulting NPs were 136.5 nm in size and slightly positive charged (4.2 mV). Under this condition, incorporation of small amounts CS-PEG (from a ratio of CS/CS-PEG of 2.5/0.1 to 2.25/0.25) led to further charge neutralization/shielding of positive charge and almost charge neutrality (FIG. 2F). Further increases in the amounts of CS-PEG led to formation of negatively charged NPs. NPs with CS-PEG (˜120 nm) were smaller than the NPs without CS-PEG (˜140 nm) (FIG. 2F).

FIG. 2G shows that there were minimal changes in the sizes of PMAOB-CP NPs after 2 weeks in PBS at RT. No obvious changes of sizes were observed either after the PMAOB-CP NPs were incubated in 50% of mouse serum for 24 h (FIG. 2G). FIG. 2H shows that free siRNA was completely degraded following treatment with RNAse III at 37° C. for 1 h. In contrast, siRNA loaded into PMAOB-CP NPs was well protected from the degradation by RNAse III (FIG. 2H). These data indicated that FuOXP/siRNA-coloaded PMAOB-CP NPs are likely to be stable in blood for sufficient time to achieve effective tumor targeting after systemic administration.

An s.c. tumor model (CT26) was used for an initial, representative optimization study. A mixture of Cy5-labeled siRNA and non-labeled siRNA (1:1, w/w) was used to prepare PMAOB-CP NPs and the in vivo distribution of the labeled siRNA in tumors was examined by fluorescence microscopy at 24 h following tail vein injection. The uptake of siRNA in liver was also examined as liver is a major organ that non-selectively takes up NPs. FIG. 3C shows the tissue distribution of Cy5-siRNA after treatment with various PMAOB-CP NPs that were prepared at a N/P ratio of 10/1 but were coated with various amounts of CS/CS-PEG, respectively. Increasing the N/P/S (CS) ratio from 1/10/1 to 1/10/2.5 was associated with a gradual increase of Cy5.5 signals in tumors and a concomitant decrease of signal in liver. (FIG. 3C). Further increases in the amounts of CS resulted in decreases of the signals in tumor and increases of signals in liver (FIG. 3C). It was then studied whether the tumor-targeting efficiency of CS-coated NPs could be further improved via incorporation of CS-PEG. In a number of studies, the N/P/S (CS) ratio was kept at 10/1/2.25 while gradually increasing the amounts of CS-PEG. increasing the ratio of CS/CS-PEG from 2.25/0.2 to 2.25/0.5 led to further increases of Cy5.5 signals in tumors while the signals in liver were decreased. (FIG. 3C). Further increases in the amounts of CS-PEG resulted in decreased signals in tumor with increased signals in the liver (FIG. 3C). Subsequent studies were conducted with PMAOB-CP NPs prepared at a N/P/S (CS)/S (CS-PEG) ratio of 10/1/2.25/0.5.

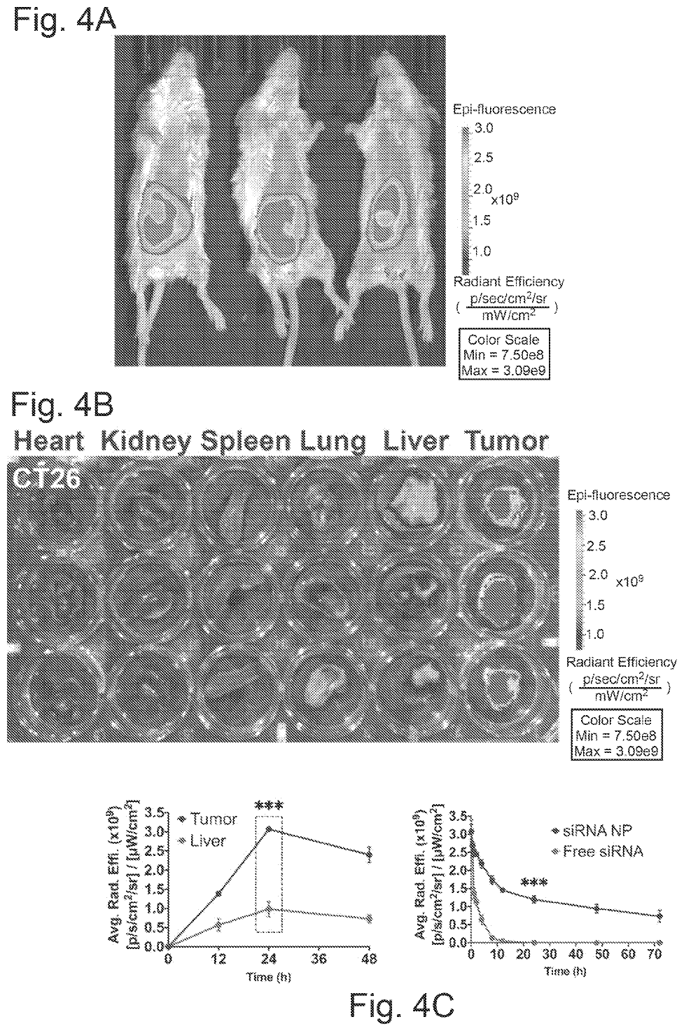

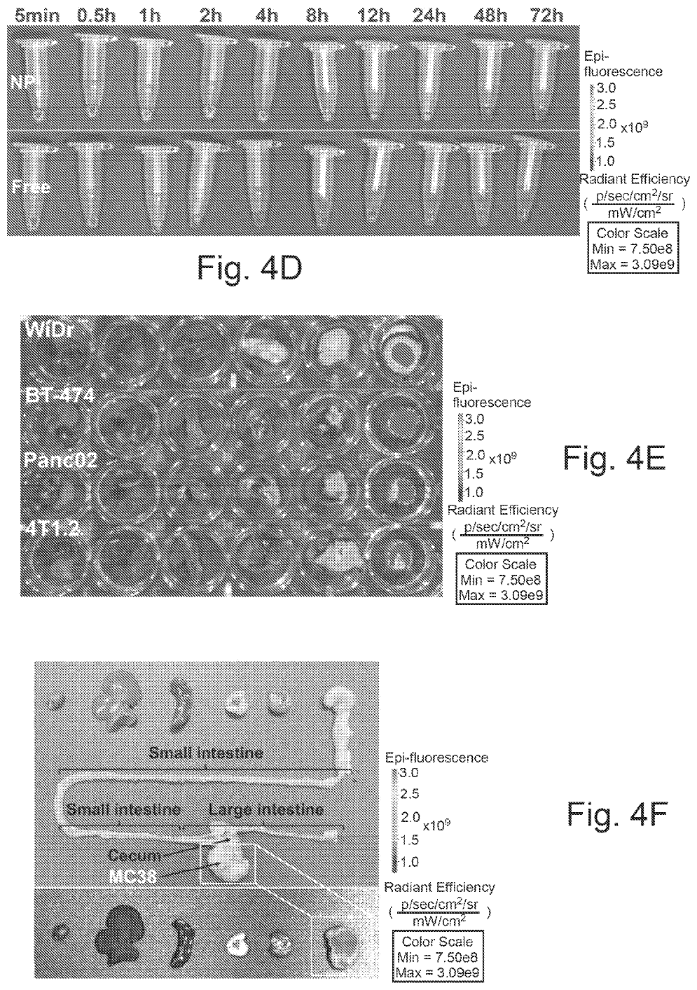

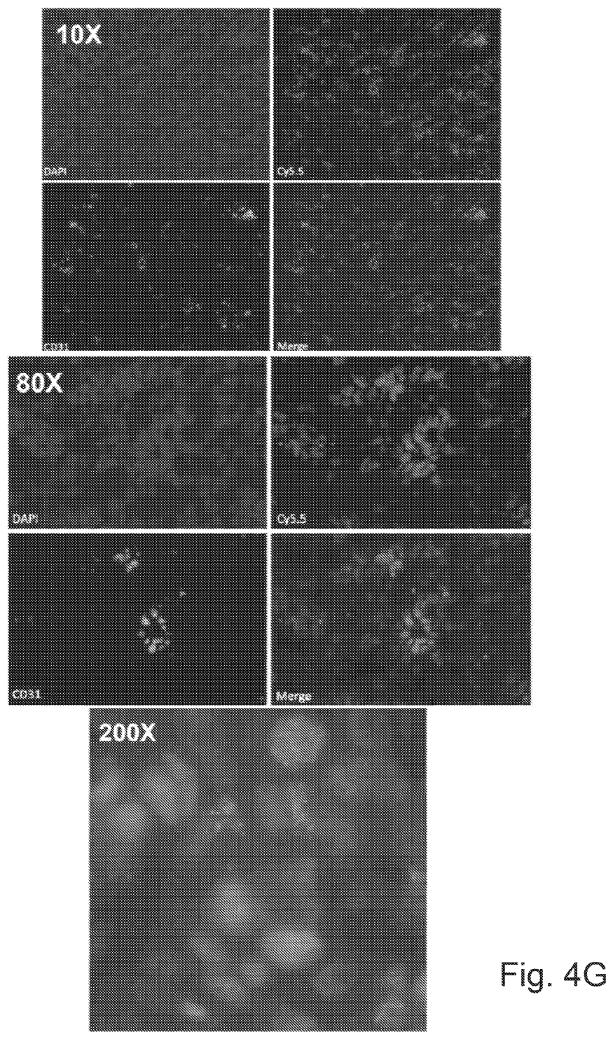

FIGS. 4A-C show the NIR images at different times following i.v. injection of Cy5-siRNA-loaded PMAOB-CP NPs. Whole-body imaging was first conducted. Tumors and major organs were then collected and subjected to ex vivo imaging. The fluorescence intensity was also quantified. Whole-body imaging showed that the CY5 signals were concentrated in the tumor areas (FIG. 4A). The ex vivo imaging data (FIG. 4B) were consistent with the results of whole-body imaging. Tumors showed the highest levels of fluorescence signals. Liver also showed obvious uptake of the NPs but the levels of Cy5 signals in liver were significant lower compared to those in tumors. Little signals were seen in other organs including heart, kidney, spleen, and lungs. FIG. 4C shows that substantial amounts of siRNA signals were detected in tumors at 12 h following i.v. injection. The levels peaked at 24 h and then slowly declined thereafter (FIG. 4C). The NPs stayed in the blood significantly longer than free siRNA (FIG. 4D).

Following the demonstration of effective tumor targeting of PMAOB-CP NPs hereof in the s.c. CT26 model, studies where then conducted to determine whether the nanocarriers hereof can also mediate selective delivery to other s.c. tumor models including human colon cancer (WiDr), human breast cancer (BT-474), murine pancreatic cancer (Panc02), and murine breast cancer (4T1.2). FIG. 4E shows that, similar to what was seen in s.c. CT26 model, Cy5 siRNA-loaded PMAOB-CP NPs were mainly concentrated at tumor tissues in all other s.c. tumor models examined. A similar result was also observed in an orthotopic murine colon cancer model (FIG. 4F). FIG. 4G shows the distribution of Cy5 siRNA in tumor sections at 24 h following i.v. administration of siRNA NPs. Widespread distribution of Cy5 signals was observed in the tumor tissues and both tumor cells and tumor ECs (CD31⁺ cells) appear to take up NPs. At high magnifications, colocalization of DAPI and Cy5 was clearly visualized, indicating that siRNA was effectively released from the endosome following intracellular delivery and reached the nucleus. Together, these data indicate that PMAOB-CP NPs can be effectively targeted to various types of cancers.

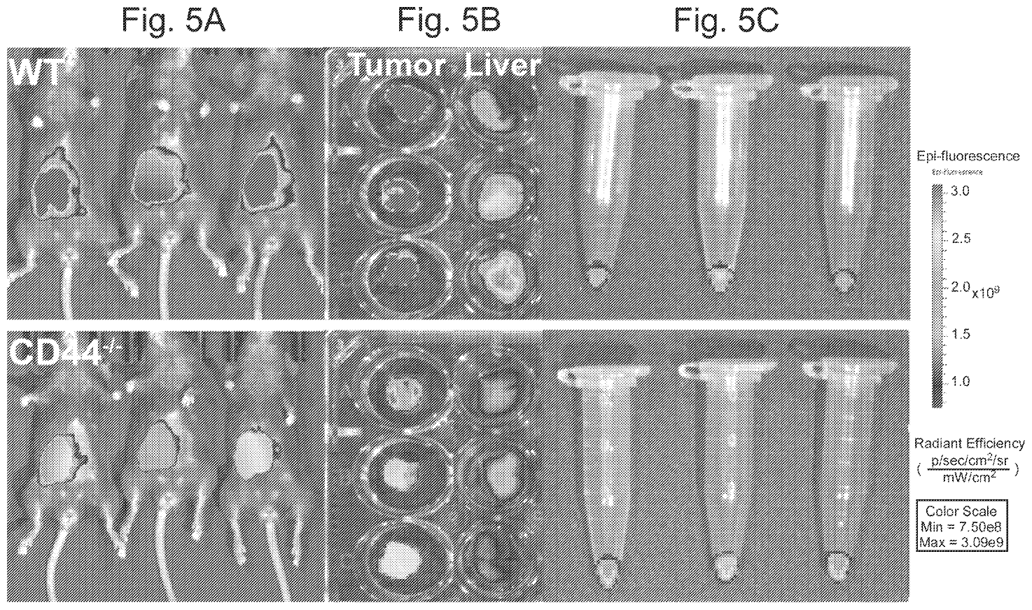

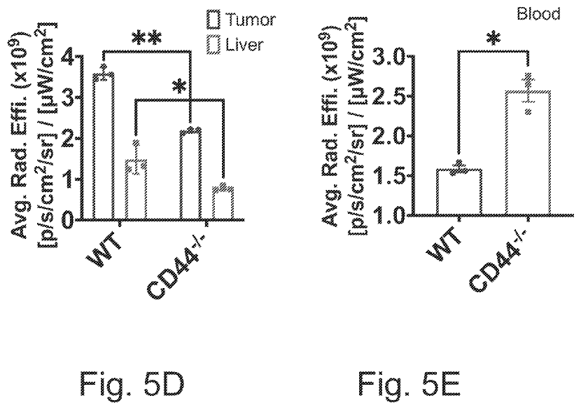

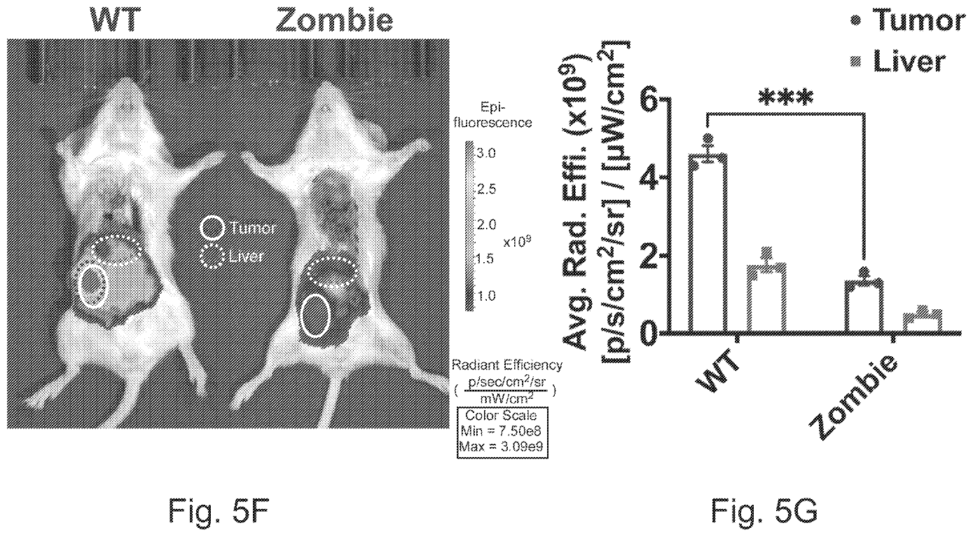

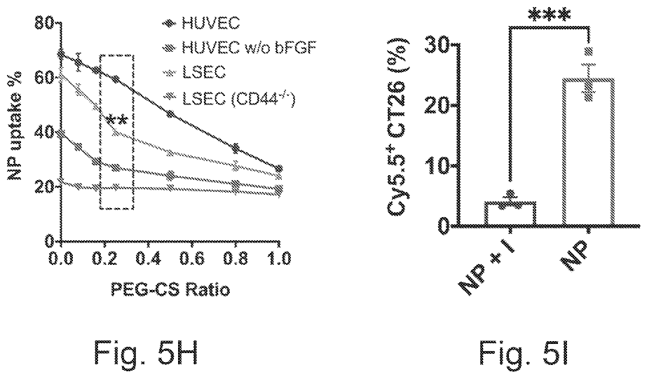

Without limitation to any mechanism, the decreased tumor uptake that was associated with increases in PEG shielding (>0.5 CS-PEG) may indicate that CS-mediated active targeting is likely to play a role in the overall tumor targeting. CS is a natural ligand of CD44 and CD44 is known to be overexpressed in both tumor cells and tumor endothelial cells (ECs). To examine whether the CD44-mediated ECs targeting plays a role in the effective tumor targeting by PMAOB-CP NPs, the NIR imaging was similarly performed in CD44^{-/31} mice and compared to the results in CD44 wild-type (WT) mice. Since CD44^{-/31} mice have a C57BL/6genetic background and are not suitable for establishing CT26 tumor, MC38 colon cancer model was used in a number of studies. As shown in FIG. 5A, Cy5 siRNA-loaded PMAOB-CP NPs were highly effective in accumulating at tumor tissues in WT mice. Their levels in tumors were significantly higher than those in liver. However, the Cy5 signals in tumor tissues were decreased significantly in CD44^{-/31} mice (FIGS. 5B and 5D). The uptake of Cy5 siRNA-loaded PMAOB-CP NPs was also decreased in the liver in CD44^{-/31} mice. The Cy5 siRNA signals in blood were increased in CD44^{-/31} mice (FIG. 5C and 5E). Such results indicate that Cy5 siRNA-loaded PMAOB-CP NPs were highly stable in the blood and that the CD44-mediated tumor ECs contributed to the overall tumor targeting. Meanwhile, CD44 in the liver sinusoidal ECs (LSECs) may also contribute to the uptake of the Cy5 siRNA-loaded PMAOB-CP NPs in the liver.

The tumor-targeting efficiency of Cy5 siRNA-loaded PMAOB-CP NPs was also evaluated in Zombie mice in which the systemic vasculature including tumor vasculature is perfused and fixed with 4% paraformaldehyde prior to tail vein injection of the NPs. In this model, the passive targeting mechanism such as EPR remains effective while the active transendothelial transport mechanism is inhibited. As shown in FIG. 5F-G, the accumulation of the Cy5 siRNA-loaded PMAOB-CP NPs in the tumors was significantly decreased in the Zombie mouse model, indicating that both active and passive targeting mechanisms contribute to the overall tumor targeting by PMAOB-CP NPs.

To further investigate the respective role of CD44 in tumor ECs and LSECs in interacting with representative PMAOB-CP NPs, the uptake of Cy5 siRNA-loaded PMAOB-CP NPs by isolated mouse LSECs and human umbilical vein endothelial cells (HUVECs) was studied. HUVECs cultured in the absence of growth factor (basic fibroblast growth factor, bFGF) stay at quiescent state and express low levels of CD44. On the other hand, HUVECs cultured in the presence of bFGF become activated and express higher levels of CD44, which are often used as a model of tumor ECs. As shown in FIG. 5H, NPs coated with CS only (without CS-PEG) were effectively taken up by both activated HUVECs and LSLECs with more NPs being taken up by activated HUVECs (69.3 vs 61.5%). It is also apparent that the quiescent HUVECs took up significantly less amounts of the NPs compared to activated HUVECs (FIG. 5H). Similar results were shown for CD44^{-/31} LSECs in comparison with WT LSECs, indicating that CD44-mediated endocytosis likely plays a role in the cellular uptake of the CS-coated NPs by both tumor ECs and LSECs.

Incorporation of CS-PEG led to decreased cellular uptake of the NPs in a PEG dose- dependent manner in both activated HUVECs and LSECs. However, CS-PEG clearly showed more impact on the uptake by LSECs compared to activated HUVECs. At the ratio of CS/CS-PEG of 2.5/0.25, the level of uptake by LSECs decreased to 39.8% while 58.7% remained for activated HUVECs (FIG. 5H). At a ratio of CS/CS-PEG of 2.5/1.0, the levels of uptake of the PMAOB-CP NPs in both types of cells decreased to comparable levels (24.4% vs 26.8%).

CD44 has been shown to be capable of mediating transcytosis. To explore a potential role of transcytosis in tumor targeting by representative PMAOB-CP NPs, a co-culture experiment was conducted with HUVECs and CT26 cells using a Transwell plate. It was apparent that CT26 cells grown in the lower chamber were effectively transfected when Cy5.5-siRNA NPs were applied to HUVECs grown in upper chamber as examined by flow analysis of Cy5.5′ CT 26 cells at 12 h (FIG. 5I). Transfection was significantly inhibited by dynasore, an endocytosis inhibitor, indicating the effectiveness of the NPs hereof in mediating transcytosis through vascular ECs.

Nanocarriers hold potentials for enhanced delivery of various types of anticancer agents, especially for codelivery of different types of therapeutics. Nanocarriers hereof are well suited to achieve codelivery of, for example, siRNA and chemotherapeutics. In addition to enhanced delivery of both types of therapeutic agents to tumors, this strategy bas the advantage of selectively delivering siRNA to those tumor cells that are exposed to chemodrugs. Embodiments of codelivery approaches hereof are, for example, effective in antagonizing mRNA that is induced in situ by co-delivered chemotherapeutic drug. Several types of amphiphilic polymers (POEG-st-Pmor and PMet-P (cdmPEG_{2K})) that were effective in co-formulating nucleic acids (plasmid and siRNA) and hydrophobic anticancer drugs (PTX and DOX) were previously studied. These carriers are highly effective in delivery to lungs including lung metastases but with limited effectiveness in selective delivery to distant solid tumors such as s.c. tumors due to their cationic surface. To facilitate codelivery of siRNA and small molecule drugs to distant solid tumors, a new type of polymer (of which PMAOB is a representative example) was developed in studies hereof with several new features (FIG. 2A). In that regard, in a number of embodiments, a lipid motif was introduced to facilitate interaction with cell membrane and improve transfection. It also helps to improve the loading of the representative chemotherapy agent FuOXP into the hydrophobic/lipophilic core. In a number of embodiments, in vivo cationic biguanidine was introduced into the polymer to enhance the interaction with siRNA. Further, in a number of embodiments, CS/CS-PEG2K was used to coat the PMAOB micelles coloaded with FuOXP/siRNA to generate PMAOB-CP NPs with neutral or slightly anionic surface, and to minimize the “nonspecific” uptake by RES. The data of studies hereof showed that the amounts and the ratio of CS/CS-PEG are adjustable in a readily determined manner for effective tumor targeting. NPs without CS-PEG showed limited effectiveness in tumor targeting as a result of substantial “non-specific” uptake by liver. In representative studied examples, increasing the ratio of CS/CS-PEG from 2.5/0.2 to 2.5/0.5 led to increased tumor accumulation with concomitant decreases in liver uptake. However, further increases in the amount of CS-PEG resulted in decreases in tumor uptake. These data, together with the data from CD44-mice strongly indicate that CD44-mediated tumor ECs-targeting/transcytosis plays a significant role in the overall tumor targeting.

CD44 is known to be overexpressed in both tumor ECs and tumor cells, and is highly effective in mediating transcytosis. Hyaluronic acid (HA) and chondroitin sulfate (CS) are endogenous polysaccharide ligands that exist in the extracellular matrix. A barrier that limits the effectiveness of HA-or CS-mediated targeting of tumor ECs is the expression of CD44 on LSECs that remove most of NPs in the circulation due to the abundance of these cells. Indeed, many reported HA-and CS-coated NPs showed extensive liver uptake with a level that is significantly higher than that in tumor. Based on the fact that tumor ECs express higher levels of CD44 than LSECs, it was hypothesized that incorporation of appropriate amounts of, for example, PEG will minimize the interaction of the NPs with LSECs without significantly compromising the binding to tumor ECs or tumor cells if the NPs manage to reach tumor cells via EPR. Use of excess amounts of PEG will block the interaction of CS-coated NPs with tumor ECs, which will result in the eventual uptake of NPs by Kupffer cells and possibly LSECs as well through CD44-independnet mechanism. The hypothesis-driven study led to the development of a representative PMAOB-CP-based nanocarrier that demonstrated high effectiveness in tumor targeting in multiple tumor models including an orthotopic colon cancer model. The levels of accumulation in tumors were significantly higher than those in liver in all tumor models examined. More importantly, the nanocarriers hereof are capable of tumor targeting through both EPR and transcytosis, which provides potential in clinical translation.

Delivery of siRNA via PMAOB-CP and other polymer-based NPs hereof leads to effective knockdown of target genes in vitro and in vivo. For example, codelivery of siRNA may significantly decrease chemotherapy-induced upregulation of certain mRNA. In addition, codelivery of a chemotherapeutic compound/agent and siRNA may provide significant improvement in antitumor activity over use NPs including only the chemotherapeutic agent in a number of cancer models. Various flow study showed increased numbers of CD45 cells, IFNγ⁺ CD8⁺ T cells and GzmB⁺ CD8+ T cells, an increased M1/M2 ratio as well as decreased Treg cells, indicating a likely role of the improved tumor immune microenvironment in the overall enhanced antitumor activity.

The hypothesis-driven studies hereof led to the development of nanocarriers that are highly effective in tumor targeting through effective tumor ECs-targeting while minimizing the LSECs-mediated liver uptake. In addition to enhanced delivery of both nucleic acid therapeutics and chemotherapeutics to tumors, this strategy has the advantage of selectively delivering nucleic acid therapeutics such as siRNA to those tumor cells that are exposed to chemodrugs. Therefore, the codelivery approach hereof may, for example, be particularly effective in antagonizing mRNA that is induced in situ by a co-delivered chemotherapeutic drug. Representative studies of codelivery of siRNA and FuOXP led to significant improvement in tumor microenvironment and enhanced antitumor activity. Targeting various immunological targets in combination with chemotherapy may provide a novel and effective immunochemotherapy for the treatment of various types of cancers including, for example, colon and pancreatic cancers.

### Experimental Methods

Materials. Dulbecco's Modified Eagle's Medium (DMEM) and trypsin-EDTA solution were bought from Sigma-Aldrich (MO, U.S.A.). Fetal bovine serum (FBS) and penicillin-streptomycin solution were purchased from Invitrogen (NY, U.S.A.). Antibodies used for flow cytometry were purchased from reputable vendors such as BioLegend and BD Biosciences.

Cells and animals. All cell lines used in this work were obtained from ATCC (Manassas, VA). S.C. models of CT26 marine CRC, HT29 human CRC, Panc02 murine PCa, PANC-1 human PCa, WiDr human CRC, BT-474 human BCa and 4T1.2 murine BCa as well as CRC orthotopic model of MC38 murine CRC were cultured in DMEM medium supplemented with 10% FBS and 1% penicillin/streptomycin at 37° C. in a humidified atmosphere with 5% CO₂.

Female C57BL/6, BALB/c and B6.129 (Cg)-Cd44^{tmlHbg}/J (CD44^{-/31} ) mice aged between 4-6 weeks were purchased from The Jackson Laboratories. All animals were housed under pathogen-free conditions according to AAALAC (Association for Assessment and Accreditation of Laboratory Animal Care) guidelines. The mouse-related experiments were performed in full compliance with institutional guidelines and approved by the Animal Use and Care Administrative Advisory Committee at the University of Pittsburgh.

RNA-seq analysis. CT26 tumor bearing BALB/c mice (n=3, ˜200 mm³) received FuOXP NP treatment with PBS as control via tail vein injection once every five days for three times. Tumors were harvested at 24 h after the last treatment. Samples were sent to Health Sciences Sequencing Core, University of Pittsburgh for RNA extraction, library construction and sequencing. RNA-seq data were aligned to mouse reference genome GRCm38 using STAR. Gene expression levels were quantified, and count expression matrices were generated using RSEM from aligned reads. Count per million was used for further analysis.

Real-time PCR. cDNA was generated from the purified RNA extracted from cultured cells, isolated tumor and stromal cells or tumor tissues using QuantiTect Reverse Transcription Kit (Qiagen, MD, U.S.A) according to the manufacturer's instructions. Quantitative real-time PCR was performed using SYBR Green Mix on a 7900HT Fast Realtime PCR System. Relative target mRNA levels were analyzed using delta-delta-Ct calculations and normalized to GAPDH.

Synthesis scheme of PMAOB. PMAO (compound 1) is sold with an average Mₙ of 30,000-50,000, which makes a polymer with ˜100-140 repeating units per polymer molecule: Mₙ is the Molecular weight of the repeating unit. For the synthesis of compound 1derivatives, calculation of the molar ratios of reactants was based on repeating monomer units A with a molecular weight of 350 g/mol. 7 grams of compound 1 (20 mmol of repeating units) was added into a 250 mL glass bottle equipped with magnetic bar and placed under an atmosphere of nitrogen. The polymer was dissolved in 150 mL of dry and degassed DMSO. A volume of 6.67 mL ethylenediamine (100 mmol) in 50 mL dry and degassed DMSO solution was then added to the solution. The solution was allowed to stir 48 hours at 160° C. under nitrogen. After the reaction, the solution was cooled down to room temperature. 1 L HCl solution (2 mol/L) was added to the DMSO solution and the precipitation was filtered and washed 3 times with water and dried in vacuum at 50° C. (compound 2). The yield is quantitative. 392 mg compound 2 (1 mmol of repeating units) and PEG_{2K}-NHS 200 mg (0.1mmol) were added into a 50 ml bottle equipped with magnetic bar and the solid was dissolved in 10 ml of dry DMSO and 1 mL TEA (triethylamine). The solution was allowed to stir 48 hours at room temperature. After the reaction, the solution was transferred to dialysis bag (MWCO 12,000-14,000) and then dialyzed in water for 24 hours. After dialysis, the solution was filtered by P5 filter paper and the filtrate was subjected to lyophilization (PEG derivative). The yield is about 10-20%. 100 mg PEG derivatives and dicyandiamide 840 mg (10 mmol) were added into a 50 ml bottle equipped with magnetic bar. The solid was dissolved in 10 mL of tert-BuOH. The solution was then reflexed and stirred for 12 hours. After the reaction, the solution was transferred to dialysis bag (MWCO 12,000-14,000) and dialyzed for 24 hours in water. After dialysis, the solution was subjected to lyophilization (compound 3: PMAOB). The yield is quantitative.

Preparation of FuOXP/siRNA-coloaded PMAOB NPs. Film hydration method was used to prepare the FuOXP-loaded PMAOB NPs. In brief, PMAOB polymer and FuOXP were mixed in dichloromethane at a ratio of 10:1 (w/w). After evaporating the solvent, nano water was added to hydrate the film to obtain the FuOXP/PMAOB NPs. The siRNA diluted with nano water is then mixed with FuOXP-loaded micelles to form FuOXP/siRNA/PMAOB complexes. Subsequent incubation with CS/CS-PEG of various ratios led to the formation of CS/CS-PEG-decorated, FuOXP/siRNA co-loaded PMAOB-CP NPs. Complexation of siRNA with PMAOB polymer will be confirmed by gel retardation assay. FuOXP loading capacity (DLC) and drug loading efficiency (DLE) of FuOXP were determined by high-performance liquid chromatography (HPLC). The particle size and zeta potential were measured by dynamic light scattering (DLS). An in vitro drug release study was conducted by dialysis method following a published protocol. FuOXP released into dialysate solutions was determined by HPLC. The colloidal stability of FuOXP/siRNA co-loaded NPs was examined in PBS (with or without 50% mouse serum) by following changes in sizes and surface charges. The integrity of siRNA following exposure to RNAse was examined by electrophoresis.

In vitro drug release. The release of FuOXP from FuOXP-loaded PMAOB-CP with or without siRNA complexation was studied using a dialysis method. Briefly, 2 mL of

FuOXP/siRNA/PMAOB-CP and FuOXP/PMAOB-CP micelles containing 1 mg of FuOXP and 10 mg of PMAOB-CP were placed in a dialysis bag (MWCO 3.5 kDa) and immersed into 40 mL of 0.1 M PBS solution containing 0.5% (w/v) Tween 80. The experiment was performed in an incubation shaker at 37° C. at 100 rpm. At selected time intervals, 10 μL solution in the dialysis bag and 1 mL medium outside the dialysis bag were withdrawn while same amount of fresh dialysis solution was added for replenishment. The concentration of FuOXP was examined by HPLC. Free FuOXP was included as control.

Whole-body near-infrared (NIR) fluorescence imaging and ex vivo imaging. Groups of 5 female BALB/c mice were each inoculated with 5×10⁵ CT26 cells at the right flank. When the tumors grew up to ˜300 mm³, the mice were i.v. administered with Cy5.5-siRNA-loaded PMAOB-CP NPs at a siRNA concentration of 1 mg/kg. At 12, 24, 36and 48 h time points, the mice were imaged by IVIS 200 system (Perkin Elmer, USA) at a 60s exposure time with excitation at 678 nm and emission at 694 nm. The tumor and various organs were excised for ex vivo imaging following a previously published protocol. Blood was withdrawn at 5 min, 0.5, 1, 2, 4, 8, 12, 48 and 72 h time points and serum samples were prepared and imaged by IVIS 200 system.

Microscopic study of tumor distribution of NPs. For in vivo tumor biodistribution study, MC38 tumor bearing mice (˜300 mm³ ) were intravenously (i.v.) injected with Cy5.5-siRNA-loaded NPs. The mice were sacrificed at 24 h post injection. Tumor frozen sections were prepared and stained with Hoechst for observation under the fluorescence microscope (BZ-X710, Japan). Blood vessels were stained with FITC-anti-CD31 antibody.

Zombie fixation and nanoparticle circulation. Zombie mice assay was conducted according to a previously published protocol. Mice were fixed using transcardiac perfusion with the TEM solution (4% formaldehyde and 0.5% glutaraldehyde in 1×PBS) for 20 min. Cy5.5-siRNA-loaded PMAOB-CP NPs at a siRNA concentration of 1 mg/mL solution was then added for circulation. The concentration of nanoparticle was the same as that in the control animal assuming 1.8 ml of blood. Each of these nanoparticle solutions was circulated in the fixed mouse at a physiologically relevant flow (6 ml min⁻¹) rate for 4 h using a peristaltic pump that alters the pressure during circulation. The mice were then imaged by IVIS 200 system for Cy5.5 detection.

Cellular uptake. Mice liver sinusoidal endothelial cells (LSECs) were isolated according to a previously published protocol from both WT C57BL/6 and B6.129 (Cg)-Cd44^{tm1Hbg}/J (CD44^{-/-}) mice. Perfused mice liver was cut out from the mice and grinded to release the cells. Cell suspension was being centrifuged several times at different speed and the suspended pellet was loaded on top of Percoll gradient. Non-parenchymal cells (NPC) were collected from the interface between the two density cushions of 25% with 50% Percoll and Kupffer cells were removed by selective adherence. LSECs were harvested by seeding the cells on collagen-coated cell-culture plastic dish. LSECs from both WT and CD44^{-/-}0 mice as well as sub-confluent HUVECs treated with or without growth factors (bFGF) were incubated with various PEG-CS ratios of Cy5.5-siRNA-loaded PMAOB-CP NPs. Cellular uptake was examined by flow cytometry after 4 h.

Therapeutic efficacy investigation of FuOXP/siRNA-coloaded NPs. In subcutaneous CT26 tumor-bearing mice. BALB/c mice (n=5, Jax) were subcutaneously injected with 5×10⁵ CT26 cells in 100 μl of serum-free DPBS at the right flank. When the tumor volume reached ˜50 mm³, mice were intravenously administered DPBS (CT), siCT NPs, siRNA NPs, FuOXP NPs, FuOXP/siCT NPs or FuOXP/siRNA NPs three times at an interval of 5 days. The tumor volumes and body weights were monitored at specific days.

In subcutaneous Panc02 tumor-hearing mice. C57BL/6 mice (n=5, Jax) were subcutaneously injected with 5×10⁵ Panc02 cells in 100 μl of serum-free DPBS at the right flank. When the tumor volume reached ˜70 mm³, mice were intravenously administered DPBS (CT), siCT NPs, siRNA NPs, FaOXP NPs, FuOXP/siCT NPs or FaOXP/siRNA NPs three times at an interval of 5 days. The tumor volumes and body weights were monitored at specific days.

Tumor-infiltrating immune cells. CT26 tumor bearing BALB/c mice received various treatments with DPBDS as control via tail vein injection once every five days for three times. Tumors and spleens were harvested at 24 h after the last treatment. Tumor-infiltrating immune cells were isolated, single cell suspensions were prepared and stained for Annexin V, CD45, CD8 (IFN-γ^{+/-} and grancyme B^{+/-}). CD4(IFN-γ^{+/-} and grantyme B^{+/-}), FoxP3 and macrophage (F4/80 and CD206) for flow cytometry analysis.

Toxicity. Body weights of mice after treatment were followed as an indication of systemic toxicity. After completing the in vivo therapy study, blood samples were collected and ALT and AST were measured by ALT/SGPT or AST/SGPT liqui-UV assay kit following manufacturer's protocols. Serum cytokine levels (TNF-α and IL-6) were determined with mouse cytokine assay kits. Tumors and major organs including heart, liver, spleen, lung and kidney were excised and fixed in PBS containing 10% formaldehyde, followed by embedment in paraffin. The paraffin embedded samples were sectioned into slices at 4 μm using an HM 325 Rotary Microtome. The tissue slices were then subjected to H&E staining for histopathological examination under a Zeiss Axiostar plus Microscope (PA, USA).

Statistical analysis. All values were presented as mean±standard error of mean (SEM). Statistical analysis was performed with two-tailed Student's t-test for comparison between two groups and one-way analysis of variance (ANOVA) for comparison between multiple groups. Results were considered statistically significant if p<0.05.

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

1. A formulation, comprising nanostructures formed from self-assembly of a plurality of amphiphilic polymers comprising cationic groups and an application added to the nanostructures, the application comprising a negatively charged CD44 ligand and a hydrophilic polymeric compound.

2. The formulation of claim 1 wherein the hydrophilic polymeric compound includes a negative charge.

3. The formulation of claim 2 wherein the hydrophilic polymeric compound comprises a conjugate of a negatively charged molecule and a hydrophilic polymer.

4. The formulation of claim 3 wherein the negatively charged molecule conjugated to the hydrophilic polymer is a CD44 ligand.

5. (canceled)

6. The formulation of claim 1 wherein the CD44 ligand is osteopontin, a collagen, a matrix metalloproteinase, chondroitin sulfate, hyaluronic acid, or a derivative thereof.

7. The formulation of claim 6 wherein the CD44 ligand is chondroitin sulfate.

8. The formulation of claim 6 further comprising a therapeutic compound associated with the nanostructures.

9. The formulation of claim 8 wherein the therapeutic compound comprises a nucleic acid which is added to the nanostructures before application of the negatively charged CD44 ligand and the hydrophilic polymer compound.

10. The formulation of claim 8 wherein the therapeutic compound comprises a hydrophobic or lipophilic therapeutic compound.

11. The formulation of claim 10 wherein the therapeutic compound is a small molecule therapeutic compound having a molecular weight below 1 kDa.

12. The formulation of claim 11 wherein the therapeutic compound has a molecular weight below 1 kDa.

13. (canceled)

14. The formulation of claim 10 further comprising a second therapeutic compound, different from the therapeutic compound, wherein the second therapeutic compound comprises a nucleic acid.

15. The formulation of claim 14 wherein the therapeutic compound is a small molecule therapeutic compound having a molecular weight below 1 kDa.

16. (canceled)

17. The formulation of claim 15 wherein the therapeutic compound is a chemotherapeutic compound.

18. The formulation of claim 9 wherein the nucleic acid comprises RNA or DNA.

19. The formulation of claim 18 wherein the nucleic acid is a gene or siRNA.

20. The formulation of claim 19 wherein the nucleic acid is siRNA.

21. The formulation of claim 1 wherein the cationic groups comprise an inherently cationic group or a group which forms a cation in vivo.

22. The formulation of claim 21 wherein the group which forms a cation in vivo is an amine group, wherein the amine group is an acyclic amine group, a cyclic amine group or a heterocyclic amine group.

23. The formulation of claim 22 wherein the amine group is selected from the group consisting of a metformin group, a morpholine group, a piperazine group, a pyridine group, a pyrrolidine group, piperidine, a thiomorpholine, a thiomorpholine oxide, a thiomorpholine dioxide, an imidazole, a guanidine, a biguanidine or a creatine.

24. The formulation of claim 3 wherein the hydrophilic polymer is selected from the group consisting of a polyalkylene oxide, a polyvinylalcohol, a polyacrylic acid, a polyacrylamide, a polyoxazoline, a polysaccharide and a polypeptide.

25. The formulation of claim 3 wherein the hydrophilic polymer is polyethylene glycol.

26. The formulation of claim 1 wherein a ratio of the negatively charged CD44 ligand to the hydrophilic polymeric compound is such that uptake of the nanostructures in the liver of a patient is maintained at a sufficiently low level to allow interaction of the negatively charged CD44 ligand with CD44 on a tumor remote from the liver.

27. The formulation of claim 1 wherein each of the plurality of amphiphilic polymers comprises a hydrophobic polymer backbone, a first plurality of pendant groups attached to the hydrophobic polymer backbone and comprising at least one of the cationic groups, and a second plurality of pendant groups attached to the hydrophobic polymer backbone and comprising at least one hydrophilic polymer.

28. The formulation 27 wherein the hydrophobic polymer backbone further comprises a pendant lipidic group.

29. The formulation of claim 27 wherein the hydrophobic polymer backbone is formed via a free radical polymerization or via a reversible-deactivation radical polymerization.

30. (canceled)

31. A method of formulating a composition comprising forming nanostructures via self-assembly of a plurality of amphiphilic polymers comprising cationic groups in an aqueous medium and adding to the nanostructures a negatively charged CD44 ligand and a hydrophilic polymeric compound.

32.-60. (canceled)

61. A method of delivering a therapeutic compound to a patient, comprising: administering a formulation comprising nanostructures formed from self-assembly of a plurality of amphiphilic polymers comprising cationic groups, the nanostructures comprising an application, the application comprising a negatively charged CD44 ligand and a hydrophilic polymer, the first therapeutic compound being associated with the formulation.

62.-117. (canceled)
